(19) United States
(12) Patent Application Publication
(10) Pub. No.: US 2006/0173238 A1
(43) Pub. Date: Aug. 3, 2006

(54) Dynamically controlled gastric occlusion device

(76) Inventor: Warren L. Starkebaum, Plymouth, MN (US)

Correspondence Address: SHUMAKER & SIEFFERT, P. A., 8425 SEASONS PARKWAY, SUITE 105, ST. PAUL, MN, 55125, US

(21) Appl. No.: 11/047,470

(22) Filed: Jan. 31, 2005

(51) Int. Cl. A61F 2/00; A61F 13/00

(52) U.S. Cl. 600/37

(57) ABSTRACT

A dynamically controlled gastric occlusion device monitors at least one physiological parameter that varies as a function of food intake and controls the degree of gastric constriction of an occluding device, such as a gastric band, based on the monitored physiological parameter. In another embodiment, the dynamically controlled gastric occlusion device controls the degree of gastric constriction based on time. The occluding device is dynamically opened or closed to either permit or prevent the passage of food through the gastrointestinal (GI) tract. By dynamically controlling the degree of gastric constriction, the device limits the ingestion of food to reduce caloric intake so that the patient loses weight while permitting the ingestion of water and the minimum amount of caloric energy necessary to prevent malnourishment.

### FIELD OF THE INVENTION

The invention relates to medical devices and methods, and in particular, to devices for the treatment of obesity.

### BACKGROUND

Various surgical techniques have been developed to treat morbid obesity. One of these techniques involves use of a gastric banding device. Gastric bands are typically constructed in the form of a hollow tube that can be inserted through a laproscopic cannula to completely encircle the upper end of the stomach and thus restrict the passage of food into the lower stomach.

There are two basic types of gastric bands: hydraulic bands and mechanical bands. Hydraulic bands are typically constructed in the form of a hollow tube that can be inserted through a laproscopic cannula to completely encircle the upper end of the stomach and thus restrict the passage of food into the lower stomach. Hydraulic bands are typically fabricated from an elastomer, such as silicone rubber. The degree of gastric constriction (the diameter of the band) depends upon the amount of fluid injected into the band. Saline is injected or withdrawn by inserting a needle into an injection port placed just under the patient's skin. Thus, the degree of gastric occlusion provided by the band, which affects the amount of food that a person can ingest, can be adjusted by varying the amount of saline in the band.

Conventional hydraulic gastric banding devices exert a continuous restricting force on the stomach to reduce the size of the upper stomach and to restrict the passage of food from the upper to the lower stomach. However, side effects and complications of conventional gastric banding devices include erosion of the exterior stomach tissue resulting from the constant pressure of the band on the exterior stomach. In addition, hydraulic bands do not offer stable banding over time. Liquid within the bands diffuses slowly through the elastomer. Hydraulic bands therefore cannot guarantee the optimal configuration of the band over time. Multiple adjustments to maintain the optimal configuration of the band are required, increasing the cost and the number of medical visits. Also, adjustment of the band requires puncture of the patient's skin, resulting in discomfort for the patient and an increased risk of infection.

With mechanical gastric bands, the degree of gastric constriction is adjusted mechanically by means of a motor embedded within the band. An external control unit wirelessly controls the motor and thus the size of the stoma opening. Such devices may be passive devices that receive their power and control signals indicative of how the band is to be adjusted wirelessly from the external control unit. Typically, adjustment of this type of band must be performed in a physician's office, thus requiring traveling to a doctor's appointment for the band to be adjusted. Adjustment of the band also requires the specialized external telemetry devices needed to deliver power and communicate constriction instructions to the gastric banding device.

### SUMMARY

In general, the invention is directed to a dynamically controlled gastric occlusion device for the treatment of obesity. The dynamically controlled gastric occlusion device monitors at least one physiological parameter that varies as a function of food intake and controls the degree of gastric constriction of an occluding device, such as a gastric band, based on the monitored physiological parameter. In another embodiment, dynamically controlled gastric occlusion device controls the degree of gastric constriction based on time. The inner diameter of the occluding device is dynamically adjusted based on time or the monitored physiological parameter to either permit or prevent the passage of food through the gastrointestinal (GI) tract.

By dynamically controlling the degree of gastric constriction, the device limits the ingestion of food to reduce caloric intake so that the patient loses weight while permitting the ingestion of water and the minimum amount of caloric energy necessary to prevent malnourishment. In addition, side effects such as erosion of the exterior stomach tissue are reduced.

In one embodiment, the invention is directed to a method comprising monitoring a physiological parameter that varies as a function of food intake, and controlling a degree of constriction of a gastric occluding device based on the monitored physiological parameter. The physiological parameter may include at least one of a blood glucose concentration, an insulin concentration, a body temperature, a distention of the stomach, a stomach acid concentration, a gastric electrical activity, and a transabdominal impedance.

In another embodiment, the invention is directed to a system comprising a sensor that monitors a physiological parameter that varies as a function of food intake, a gastric occluding device positioned to restrict food intake by a patient, and a control unit that controls a degree of constriction of the gastric occluding device based on the monitored physiological parameter.

In another embodiment, the invention is directed to a system comprising a gastric occluding device positioned to restrict ingestion of food by a patient, and a control unit that controls a degree of constriction of the gastric occluding device based on time.

In another embodiment, the invention is directed to a computer-readable medium comprising instructions that cause a processor to monitor a physiological parameter that varies as a function of food intake, and control a degree of constriction of a gastric occluding device based on the monitored physiological parameter.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram illustrating an example embodiment of a dynamically controlled gastric occlusion device positioned with respect to a patient.

FIG. 2 is a block diagram illustrating an example embodiment of the dynamically controlled gastric occlusion device shown in FIG. 1.

FIG. 3 is a diagram illustrating another example embodiment of a dynamically controlled gastric occlusion device positioned with respect to a patient.

FIG. 4 is a block diagram illustrating an example embodiment of the dynamically controlled gastric occlusion device shown in FIG. 3.

FIG. 5 graph showing an exemplary sensed physiological parameter over a period of time.

FIG. 6 is a flow diagram illustrating a technique for controlling the degree of constriction of a dynamically controlled gastric occlusion device based on a sensed physiological parameter.

FIG. 7 is a flow diagram illustrating a technique for controlling the degree of constriction of a dynamically controlled gastric occlusion device based on time.

### DETAILED DESCRIPTION

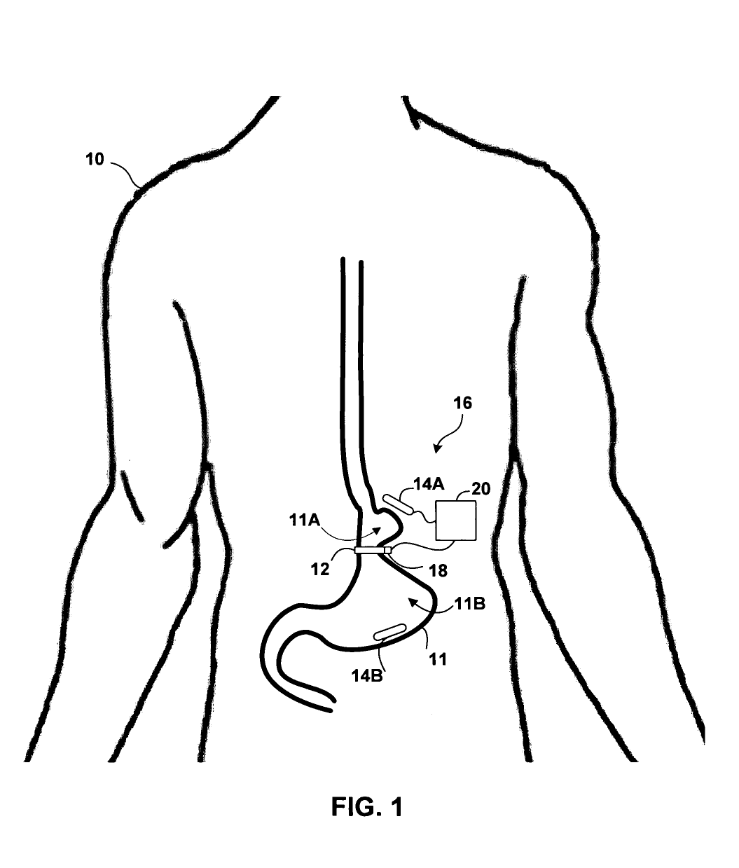

FIG. 1 is diagram illustrating a view of a torso of a patient 10, in which stomach 11 is visible. A dynamically controlled gastric occlusion device 16 monitors at least one physiological parameter that varies as a function of food intake and controls the degree of gastric constriction of an occluding device 12 based on the monitored physiological parameter. In another embodiment, dynamically controlled gastric occlusion device 16 controls the degree of gastric constriction based on time. The inner diameter of occluding device 12 dynamically increases or decreases based on time or the monitored physiological parameter to either permit or restrict the passage of food through the gastrointestinal (GI) tract. The occluding device 12 restricts passage of food (and as a result, may dramatically suppress the appetite) by creating a small stomach pouch in the upper stomach 11A and restricting a size of a stoma opening into the lower stomach 11B. By dynamically controlling the degree of gastric constriction, device 16 limits the ingestion of food to reduce caloric intake so that the patient loses weight while permitting the ingestion of water and the minimum amount of caloric energy necessary to prevent malnourishment.

Dynamically controlled gastric occlusion device 16 includes an occluding device 12, such as a gastric band, and control circuitry 18 for controlling the degree of gastric constriction, and thus the size of the stoma opening from the stomach, provided by gastric occluding device 12. In the case of a gastric band, decreasing the inner diameter of the gastric band increases the degree of gastric constriction provided by the band. At least one sensor, such as sensor 14A and/or 14B, monitors a physiological parameter that varies as a function of food intake. An implanted control module 20 monitors and analyzes the sensed physiological parameters and dynamically controls adjustment of the gastric band 12 based on time or based on the monitored physiological parameter. When the physiological parameter so indicates, control module 20 generates and transmits an adjustment control signal to control circuitry 18 within occluding device 12. Control circuitry 18 receives the adjustment control signal and adjusts occluding device 12 accordingly. For example, if the physiological parameter satisfies criteria indicative of food intake, control module 20 generates an adjustment control signal instructing control circuitry 18 to adjust gastric band 12 so that the size of the stoma opening is decreased. As another example, control module 20 may generate an adjustment control signal instructing control circuitry to adjust the gastric band so that the stoma opening is increased at pre-selected mealtimes, after a predetermined period of time, or after a physiological parameter returns to a threshold level.

Occluding device 12 may be any type of gastric occluding device, such as an electromechanical gastric band, inflatable balloon placed within the patient's stomach, or other type of gastric occluding device designed to restrict or limit food intake. Control circuitry 18 may be any circuitry designed to adjust the degree of constriction applied by the occluding device 12. For example, control circuitry 18 may be a micro motor designed to adjust the degree of constriction provided by an electromechanical gastric band, such as the telemetric adjustable gastric banding device described in published PCT Application PCT/EP03/02324, filed Mar. 6, 2003, published Sep. 18, 2003, the content of which is incorporated herein by reference in its entirety. Occluding device 12 may also be any other type of mechanically adjustable gastric band. Because the occluding device is a mechanical device, the geometric configuration and the degree of gastric constriction are stable over time.

In another embodiment, control circuitry 18 may include a motor/pump unit and a fluid reservoir that are also implanted in the patient. In this case, the occluding device may be hydraulically operated in which fluid is pumped by the motor/pump unit from the reservoir through a conduit to the occluding device to reduce the size of the stoma opening based on the monitored physiological parameter or based on time. Additionally, the motor/pump unit may pump fluid from the occluding device back to the reservoir to enlarge the size of the stoma opening based on the monitored physiological parameter or based on time.

Although in FIG. 1 occluding device 12 is shown positioned around the top end (fundus) of the stomach, the device may also be placed at another position around the stomach, vertically or in any other position designed to restrict the size of the stoma (outlet from the stomach). In addition, although the embodiment of FIG. 1 shows a gastric band associated with the stomach, a different type of occluding device could also be used, such as an inflatable balloon placed inside the stomach, which gives the patient the constant feeling of being sated. Also, the device need not necessarily be associated with the stomach, but may be associated with some other portion of the gastrointestinal tract, such as the mouth, the esophagus, the lower esophageal sphincter, or the intestines. It shall therefore be understood that any type of occluding device designed to reduce or limit food intake could be used in place of a gastric band.

Parameters such as blood glucose or insulin concentration, core body temperature, distention of the stomach, and pH level of the stomach are examples of parameters that vary as a function of food intake. In one embodiment of the present invention, one or more physiological parameters that vary as a function of food intake are used to adjust the degree of gastric constriction provided by device 16.

To accomplish this, one or more of sensors 14A and/or 14B (generally referred to as “sensor(s) 14”) monitor physiological parameters that vary as a function of food intake. Sensor(s) 14 may be associated with the stomach, as shown in FIG. 1, or may be associated with some other portion of the gastrointestinal (GI) tract, such as the mouth, esophagus, intestines, etc., or may measure other related physiological parameter such as blood glucose concentration or various temperatures of or inside the body. In FIG. 1, sensor 14A is implanted in the body of patient 10, but is external to stomach 12. Sensor 14A is communicatively coupled with control module 20, e.g., by one or more implantable wire leads. Sensor 14B, by contrast, is deployed inside stomach 11, and may communicate with control module 20 wirelessly. Alternatively, in some embodiments, sensor 14B may be coupled to control module 20 via one or more implantable wire leads that penetrate the stomach wall. It shall be understood that other types of sensors may also be used, that the invention is not limited to deployment of two sensors, and that the invention is not limited to deployment of sensors at the sites shown in FIG. 1.

Sensor(s) 14 may be any sensor that senses or responds to any physiological parameter that varies as a function of food intake. In some embodiments, sensor(s) 14 includes one or more electrodes to detect gastric electrical activity, transabdominal impedance, or other electrical indicators of stomach activity. Sensor(s) 14 may also include ultrasound sensors, motion sensors, or other sensors to detect physical movement or motion of the stomach. In other embodiments, sensor(s) 14 includes a chemical sensor that detects blood glucose, stomach acid, or other chemical indicators of stomach activity. In other embodiments, sensor(s) 14 may include an indwelling temperature sensor, such as a thermocouple or temperature sensitive resistor, to detect changes in core body temperature, or other temperature indicators of stomach activity. In further embodiments, sensor(s) 14 includes one or more mechanical sensors to detect motion of stomach 11, distention of stomach 11, or other mechanical indicators of stomach activity. The invention is not limited to mechanical, chemical, electrical, or temperature sensors however, but includes other types of sensor as well, such as auditory sensors, or any other type of sensor capable of monitoring any type of parameter that varies as a function of food intake. In addition, the sensors need not be located with respect to the stomach, but may be located with respect to the mouth, esophagus, or other areas of the digestive tract.

Physiological parameters sensed by sensor(s) 14 are supplied to control module 20. In the embodiment shown in FIG. 1, control module 20 is implanted within the body of patient 10. In other embodiments, control module 20 may be located outside of the patient's body. Control module 20 may measure, analyze, and track the parameter over time. For example, control module 20 may measure and track the amplitude of the parameter, the duration of the parameter, the intensity or concentration of the parameter, the rate of change of the parameter, or other qualities. Control module 20 controls the degree of gastric constriction of occluding device 12 based on the monitored physiological parameters.

When sensor 14B comprises a mechanical sensor that senses distension of stomach 11, control module 20 measures and records information concerning the sensed distension and stores information based on the measurement. The information may include information concerning the timing of the distension, the rate of distension, the magnitude of the distension, and the like. Control module 20 compares the information concerning the monitored physiological parameter and compares it to criteria indicative of food intake. If the monitored physiological parameter meets the criteria indicative of food intake, control module 20 generates and transmits a control signal to control circuitry 18 to adjust (i.e., increase) the degree of constriction provided by gastric band 12. By reducing the size of the stoma opening, the dynamically controlled gastric occlusion device 16 prevents or restricts the patient 10 from further ingestion of food, or reduces appetite by creating a small stomach pouch in upper stomach 11A.

In addition, device 16 may also dynamically cause the degree of constriction to be reduced (i.e., cause the size of the gastric band to be increased or “opened”) for a variable period of time and then close at pre-selected mealtimes throughout a 24-hour period. For example, device 16 may be set to open for a predefined duration at breakfast, lunch, and dinner, or other pre-selected times throughout the day. This allows the patient a relatively brief amount of time to consume food at normal mealtimes throughout the day. Since the rate of food ingestion is limited, this mechanism may help reduce caloric intake and lead to weight loss in the patient. When a decrease in the degree of gastric constriction is indicated, device 16 allows the patient to ingest a small amount of food and water to support the minimum caloric requirements of patient 10.

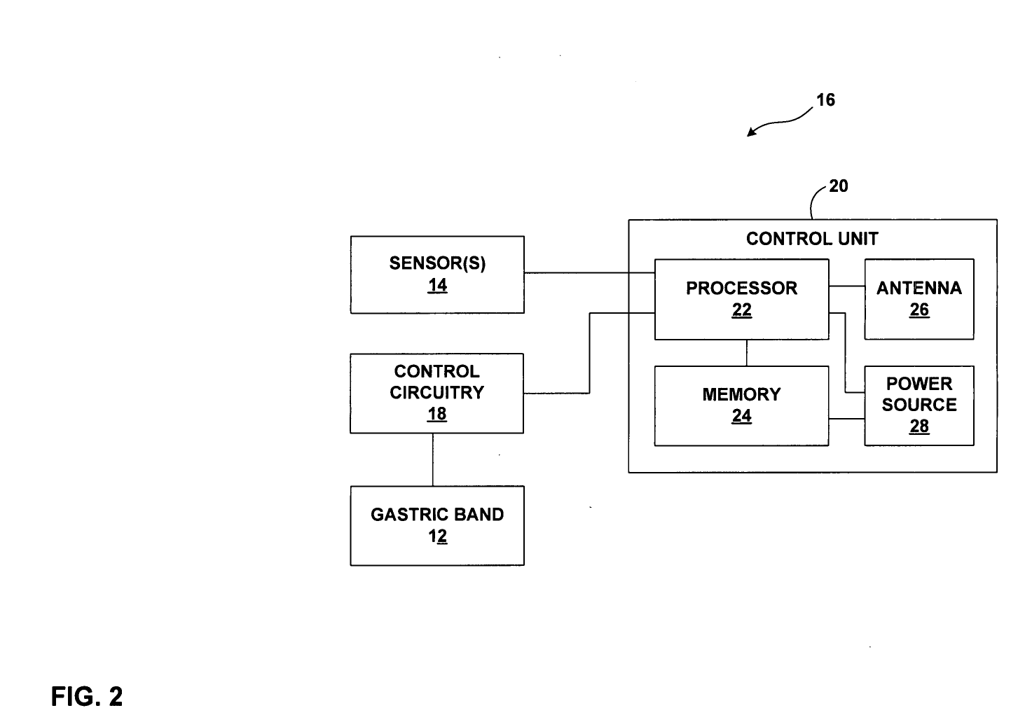

FIG. 2 is a block diagram illustrating an example embodiment of the dynamically controlled gastric occlusion device 16. In this embodiment, device 16 is implanted within the body of patient 10 (not shown). Power is provided by a power source 28, such as a battery or other suitable power source. In embodiments where control module 20 is located outside of the patient, power could be provided on board within or attached to occlusion device 12. An antenna 26 allows communication via RF telemetry to external devices. For example, during an office visit, a physician may download data stored in memory 24 from control unit module 20 to another device or computer. This allows the physician to gather, monitor, and review information concerning operation of the patient's device 16. This may further allow the physician to assess the course of treatment and determine whether any adjustments are necessary. In the case where adjustments are desired, the physician or other user may remotely program control unit 20 to correspond to a new course of treatment.

Sensor(s) 14 is positioned with respect to patient 10 to sense physiological parameters that vary as a function of food intake. Control module 20 receives sensed signals concerning the monitored physiological parameters from sensor(s) 14. Processor 22 processes and analyzes the received signals to obtain measurements of the physiological parameter of interest or of characteristics of the physiological parameter.

The received signal may be converted to digital values and stored in memory 24. The corresponding data, such as the measurements and other information obtained via and analysis of the received physiological parameter or characteristic of the physiological parameter, may also be stored in memory 24. Memory 24 may include any form or volatile memory, non-volatile memory, or both. In addition to data sensed via sensor(s) 14, memory 24 may store records concerning measurements of detected physiological parameters, criteria indicative of food intake or criteria for reducing the degree of constriction provided by device 16, communications to an external device, or other information pertaining to operation of external control module 20. Memory 24 may also store information about patient 10. In addition, processor 22 is typically programmable, and programmed instructions reside in memory 24.

Processor 22 determines whether to generate and transmit an adjustment control signal to occluding device 12 based upon the physiological parameter. For example, processor 22 may compare a physiological parameter, or one or more characteristics of a physiological parameter, to criteria indicative of food intake, and may generate an adjustment control signal when the criteria is satisfied. The adjustment control signal may be transmitted to control circuitry 18, thus for example, directing control circuitry 18 to increase the amount of gastric constriction applied by the occluding device 12.

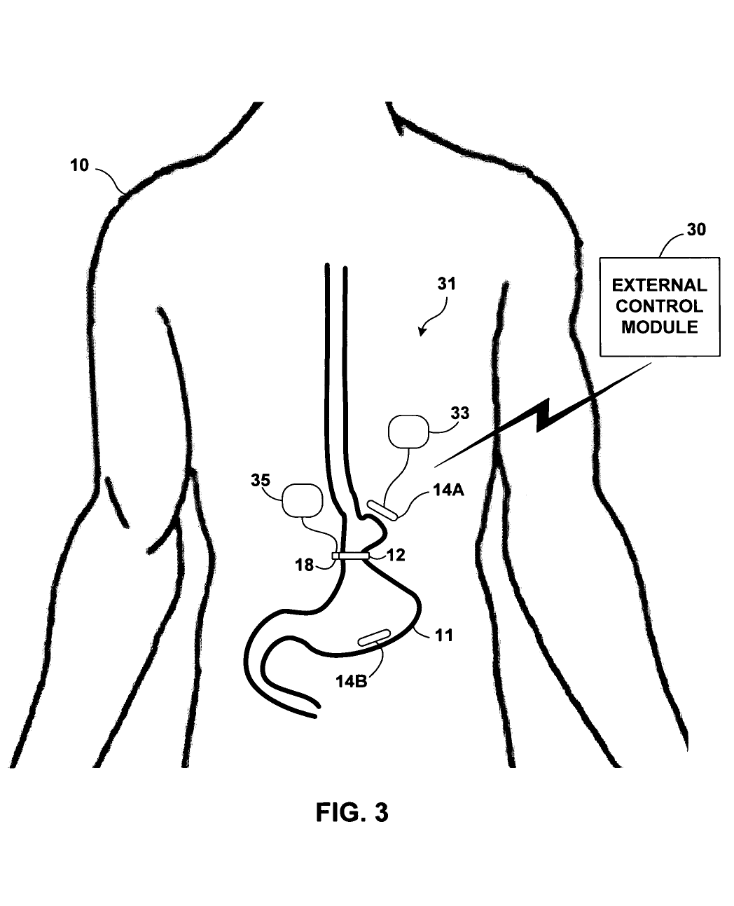

FIG. 3 is a diagram illustrating another example embodiment of a dynamically controlled gastric occlusion device 31 positioned with respect to a patient 10. In this embodiment, an external control module 30 controls the degree of constriction of occluding device 12 based on at least one physiological parameter that varies as a function of food intake, or based on time. Sensor(s) 14 sense physiological parameters that vary as a function of food intake and wirelessly communicate with external control module 30 via antenna 33. External control module wirelessly communicates with control circuitry 18 via antenna 35 to control the degree of the constriction of gastric band 12.

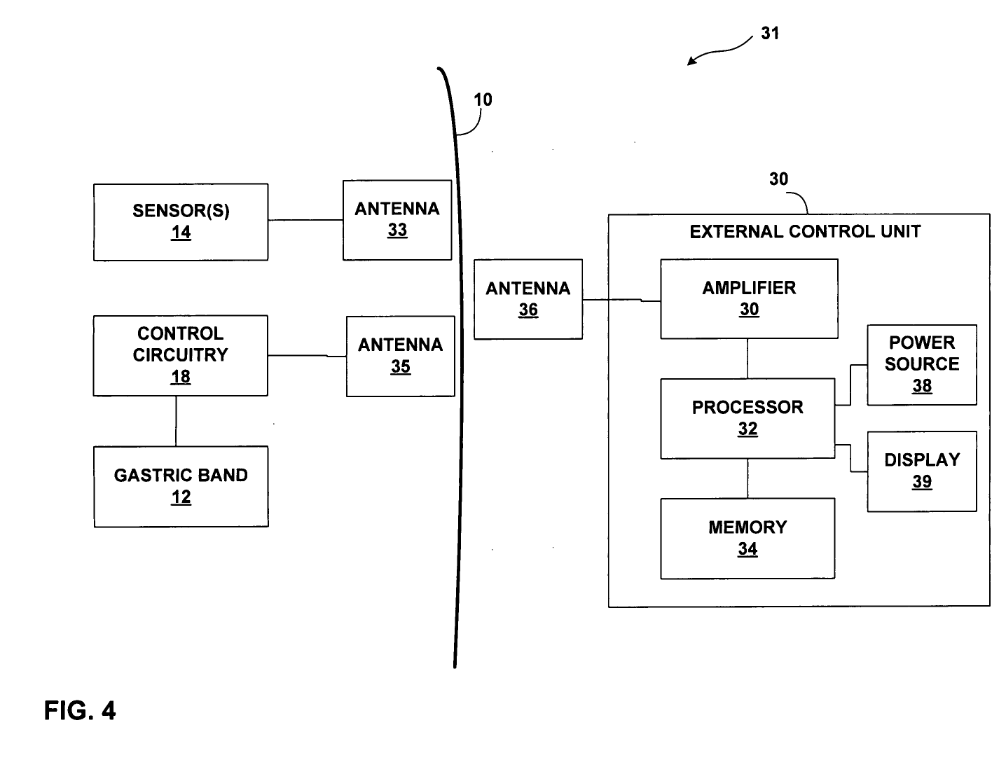

FIG. 4 is a block diagram illustrating an example embodiment of the dynamically controlled gastric occlusion device 31 of FIG. 3. Sensor(s) 14 is positioned with respect to patient 10 to sense physiological parameters that vary as a function of food intake. Sensor(s) 14 wirelessly communicate the sensed physiological parameters via antenna 33. External control module 30 receives sensed signals via antenna 36. An amplifier 30 amplifies and filters the received signals and supplies the signals to a processor 32. Processor 32 processes the received signals, and analyzes the physiological parameter of interest in the manner described above to determine whether the degree of gastric constriction should be adjusted.

The wireless communication may be achieved using RF telemetry or other type of wireless communication. External control unit 31 may be powered by an internal power source 38, such as a battery, or may be powered externally.

Power and commands to operate control circuitry 18 and adjust gastric band 12 are sent from the external control unit using electromagnetic coupling. To receive the telemetric energy, control circuitry 18 is connected to antenna 35, which is placed just under the patient's skin. When the external antenna 36 is placed near the location of implanted antenna 35, external control unit 30 sends the appropriate control signals to control circuitry 18 to adjust the diameter of occluding device 12. In one embodiment, the gastric band may be adjusted from an inside diameter of 15 millimeters to 35 millimeters, for example. Similarly, to receive the sensed physiological parameters from sensor(s) 14, a sensor antenna 33 is connected to sensor(s) 14 and is also placed just under the patient's skin. When external antenna 36 is brought near the location of sensor antenna 33, antenna 36 may receive the sensed physiological parameter. In another embodiment, control circuitry may receive power from an implanted battery that is externally recharged.

Like the embodiment shown in FIG. 2, the received signal may be converted to digital values and stored in memory 24. The corresponding data, such as the measurements and other information obtained via and analysis of the received physiological parameter or characteristic of the physiological parameter, may also be stored in memory 34. Memory 34 may include any form or volatile memory, non-volatile memory, or both. In addition to data sensed via sensor(s) 14, memory 34 may store records concerning measurements of detected physiological parameters, criteria indicative of food intake or criteria for reducing the degree of constriction provided by device 31, communications to an external device, or other information pertaining to operation of external control module 30. Memory 34 may also store information about patient 10. In addition, processor 32 is typically programmable, and programmed instructions reside in memory 34. External control module 30 may also be configured to wirelessly transmit information about the history or status of the device to another external device, such as a physician's computer or other device, as described above.

In some embodiments, patient 10 may carry external control module 30 on his person. External control module 30 may also include a display 39 that presents information to patient 10 based on the monitored physiological parameters that vary as a function of food intake. The information may be presented visually, audibly, tactilely, or in any other manner. External control module 30 may be a device dedicated to controlling the occluding device and presenting information pertaining to stomach activity, or external control module 30 may be a general purpose device such as a pager, cellular telephone, or personal digital assistant (PDA). In one embodiment, external control module 30 may also present patient 10 with information about the sensed physiological parameter by, for example, sounding an alarm and displaying a message. In response to the message, patient 10 can change his behavior, such as by discontinuing eating until the distension has subsided.

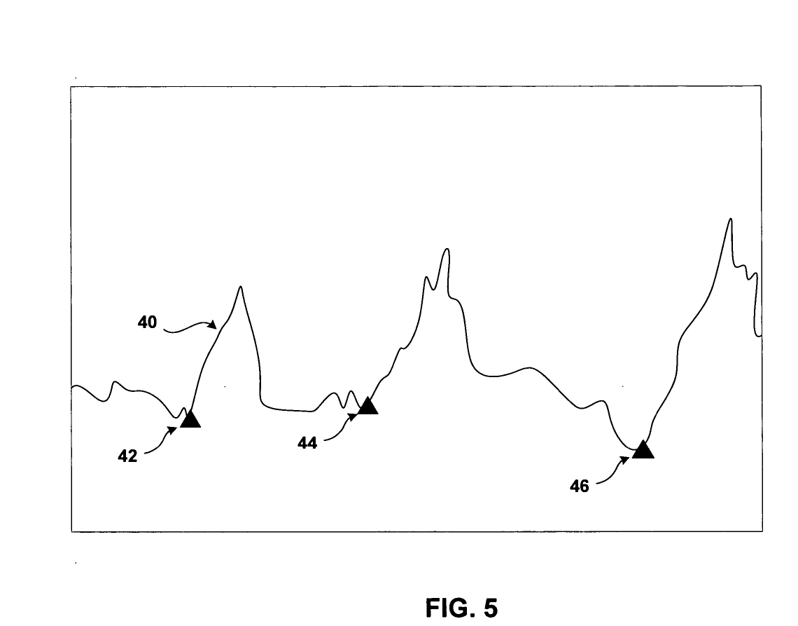

FIG. 5 is a graph showing variation of an exemplary physiological parameter over time. FIG. 5 shows an example graphical representation 40 of blood glucose for patient 10 sensed by sensor(s) 14 over a period of time. In a normal person, blood glucose rises after the ingestion of food as food is digested and nutrients are absorbed in the small intestine. The signal from a glucose sensor may be used to trigger the closing of the occluding device 12, thus preventing or restricting further food ingestion by the patient. FIG. 5 is demonstrative and does not represent actual measured data. Sensor(s) 14 may sense blood glucose levels chemically, optically, with infrared light, or using any other sensing technique.

In FIG. 5, the blood glucose level is initially stable and at a baseline level. After consumption of meals, as indicated by reference numerals 42, 44 and 46, sensor(s) 14 detects an increase in blood glucose. Processor 22 of control module 20 (or processor 32 of external control module 30) measures a characteristic of the physiological parameter, such as the amplitude, rate of change, duration of elevated glucose level, or any other characteristic. Further, processor 22 compares the measured characteristic to criteria indicative of food intake stored in memory 24. When the measured characteristic satisfies the criteria indicative of food intake, processor 22 generates and transmits an adjustment control signal to control circuitry 18. The adjustment control signal causes control circuitry 18 to decrease the diameter of the gastric band 12 in a way that further restricts the size of the stoma opening in the stomach.

The criteria for adjusting the degree of gastric constriction may vary from patient to patient. For some patients, a sharp increase in blood glucose may result in a decrease of the diameter of the gastric band. In other patients, a sharp increase is of less concern than a high amplitude or peak value of the blood glucose concentration. In a further set of patients, the duration of elevated blood glucose may be of special concern. Through receipt and analysis of a sensed physiological parameter, processor 22 may measure and track a variety of characteristics of a single physiological parameter.

In addition, processor 22 may measure a characteristic of one physiological parameter as a function of another physiological parameter. There is a relationship, for example, between the blood glucose levels following a meal and the caloric content of the meal. By analysis of blood glucose levels, processor 22 can estimate the caloric intake of patient 10. In an obese patient, an estimate of caloric intake may also be used to determine whether the degree of gastric constriction should be adjusted.

In the event the measured characteristic satisfies the criteria indicative of food intake, processor 22 generates an adjustment control signal to gastric band 12. By decreasing the diameter of gastric band 12 to further restrict the size of the stoma opening, the patient is prevented, or at least restricted, from further ingestion of food.

Control module 20 may continue to monitor the physiological parameter as indicated by the arrow going back to reference numeral 50. For example, control module 20 may monitor the physiological parameter to determine if and when the diameter of occluding device 12 should be increased. For example, control module 20 may generate an adjustment control signal to allow an increase in the diameter of gastric band 12 to allow patient 10 to consume a small amount of food. Control module 20 may allow the diameter of gastric band 12 to be increased after the sensed physiological parameter returns to a certain point (for example, after blood glucose returns to its baseline level or to within some percentage of its baseline level) or after a defined period of time since the degree of gastric constriction was increased.

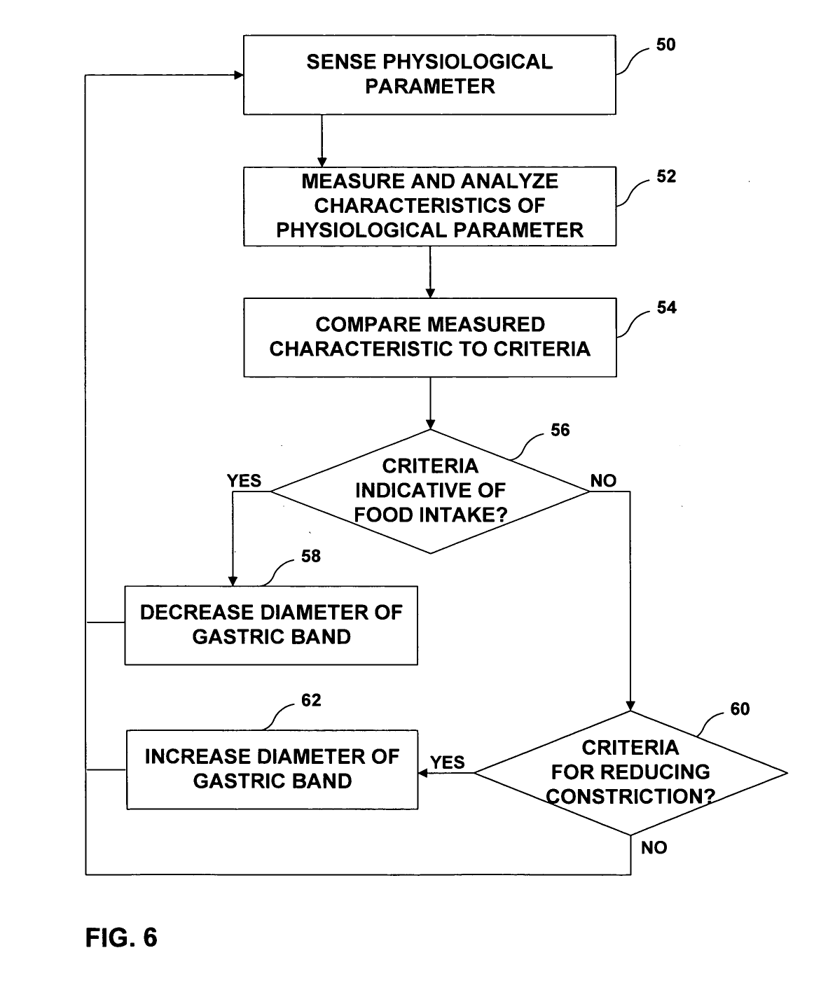

FIG. 6 is a flow diagram illustrating a technique for dynamically controlling the degree of constriction based on one or more physiological parameters that vary as a function of food intake. Processor 22 (or processor 32) receives data concerning at least one physiological parameter that varies as a function of food intake from sensor(s) 14 (50). Sensor(s) 14 may respond to any of several electrical, mechanical, chemical, temperature, or other physiological parameters.

Processor 22 processes the data received from sensor(s) 14 and measures one or more characteristics as a function of the sensed physiological parameter (52). The measured characteristic can be a characteristic of the physiological parameter itself, such as the concentration of blood glucose, core body temperature or the magnitude of stomach distension. The measured characteristic may also be a characteristic of the physiological parameter over time, such as rate of change of the parameter, duration that the parameter exceeds a threshold level, or other such characteristic. The measured characteristic can also be a characteristic of a related physiological parameter, such as a measurement of caloric intake as a function of blood glucose levels.

Processor 22 compares the measured characteristic to criteria (54) stored in memory 24. The criteria includes criteria indicative of food intake. When the measured characteristic of the physiological parameter satisfies the criteria indicative of food intake, processor 22 generates and transmits an adjustment control signal to control circuitry 18 to decrease the diameter of gastric band 12. When the measured characteristic does not satisfy the criteria indicative of food intake, processor 22 may continue to monitor the physiological parameters. In some implementations, a measurement will “satisfy” the criteria indicative of food intake when the measurement is above a defined threshold, and in other implementations, the measurement will “satisfy” the criteria indicative of food intake when the measurement is below a defined threshold, depending upon the precise physiological parameter being measured.

Processor 22 may also compare the measured characteristic to criteria for reducing the degree of gastric constriction by increasing the diameter of gastric band 12. Such criteria could be, for example, criteria indicative of a return to pre-food intake levels. For example, the diameter of gastric band 12 may be increased after the measured characteristic returns to its baseline level, to within some percentage of its baseline level, or after a specified period of time. If the measured characteristic satisfies the criteria for reducing the degree of constriction (60), device 16 may increase the diameter of gastric band 12 (62).

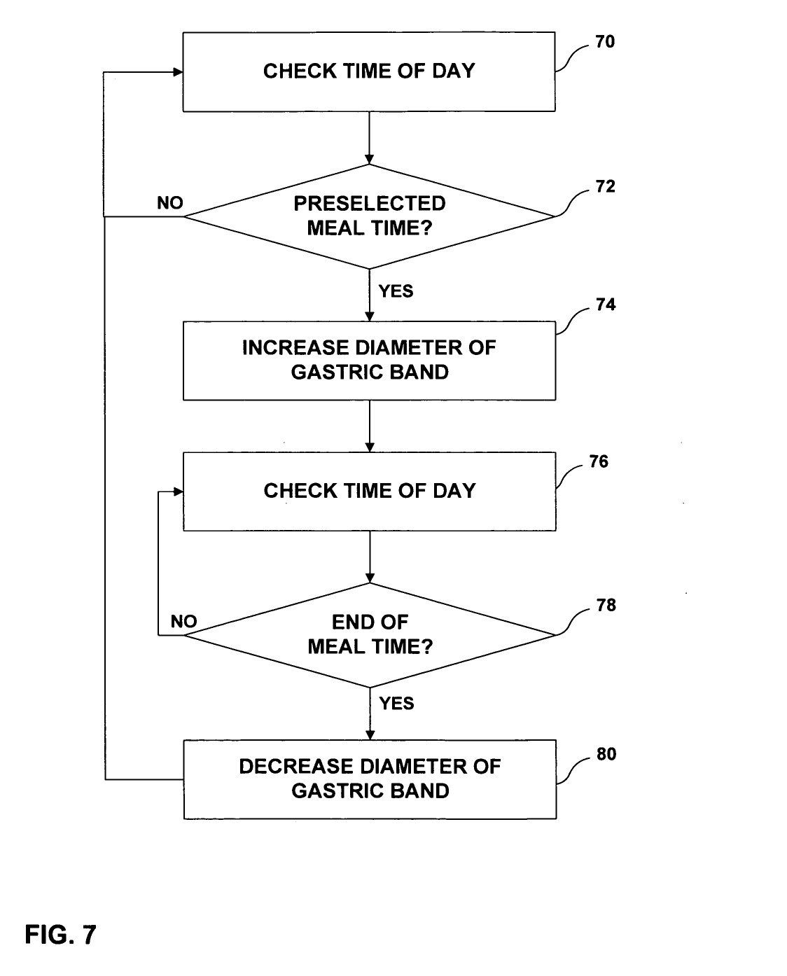

FIG. 7 is a flow diagram illustrating a technique for dynamically controlling the degree of constriction based on time. In this embodiment, device 16 is controlled by a timer such that the occluding device (for example, gastric band 12) opens at pre-selected meal times throughout 24 hour period, and then closes after a predefined period of time. For example, the device may be set to open for a period of time at breakfast, lunch, dinner, and/or any other pre-selected mealtime. The duration may be uniquely determined for each patient, but may be anywhere between 2 and 20 minutes, for example. This allows the patient a relatively brief amount of time to consume food at normal mealtimes throughout the day. Because the rate (duration) of food consumption is limited, this embodiment may reduce overall food intake, leading to a decreased caloric intake and resulting weight loss.

In the embodiment shown in FIG. 7, processor 22 (or processor 32) continuously checks the time of day (70) to determine arrival of a pre-selected meal time (72). If one of the pre-selected meal times has arrived, device 16 adjusts gastric band 12 to increase its inner diameter, i.e., to reduce the degree of gastric constriction, and allow ingestion of food (74). Processor 22 then checks the time of day (76) until the end of the pre-selected meal time (78). At this point, processor 22 causes the occluding device to be adjusted to decrease its inner diameter, i.e., to increase the degree of gastric constriction and thus prevent or restrict further ingestion of food (80). Processor 22 continues this control loop, opening and closing the gastric band 12 at pre-selected meal times throughout each 24-hour period.

In one embodiment, the invention may encompass one or more computer-readable media comprising instructions that cause a processor, such as processor 22 or processor 32, to carry out the methods described above. A “computer-readable medium” includes but is not limited to read-only memory (ROM), random access memory (RAM), non-volatile random access memory (NVRAM), electrically erasable programmable read-only memory (EEPROM), flash memory a magnetic hard drive, a magnetic disk or a magnetic tape, a optical disk or magneto-optic disk, a holographic medium, or the like. The instructions may be implemented as one or more software modules, which may be executed by themselves or in combination with other software. A “computer-readable medium” may also comprise a carrier wave modulated or encoded to transfer the instructions over a transmission line or a wireless communication channel.

The instructions and the media are not necessarily associated with any particular computer or other apparatus, but may be carried out by various general-purpose or specialized machines. The instructions may be distributed among two or more media and may be executed by two or more machines. The machines may be coupled to one another directly, or may be coupled through a network, such as a local access network (LAN), or a global network such as the Internet.

The invention may also be embodied as one or more devices that include logic circuitry to carry out the functions or methods as described herein. The logic circuitry may include a processor that may be programmable for a general purpose or may be dedicated, such as microcontroller, a microprocessor, a Digital Signal Processor (DSP), an Application Specific Integrated Circuit (ASIC), a field programmable gate array (FPGA), and the like. One or more of the techniques described herein may be partially or wholly executed in software. For example, a computer-readable medium may store or otherwise comprise computer-readable instructions, i.e., program code that can be executed by a processor to carry out one of more of the techniques described above.

The preceding specific embodiments are illustrative of the practice of the invention. It is to be understood, therefore, that other embodiments known to those skilled in the art or disclosed herein may be employed without departing from the invention or the scope of the claims. For example, the present invention further includes within its scope methods of making and using systems as described herein. Furthermore, the invention includes embodiments that use techniques to sense physiological parameters in addition to those specifically described herein.

Many embodiments of the invention have been described. Various modifications may be made without departing from the scope of the claims. These and other embodiments are within the scope of the following claims.

1. A method comprising:
monitoring a physiological parameter that varies as a function of food intake; and
controlling a degree of constriction of a gastric occluding device based on the monitored physiological parameter.

2. The method of claim 1, wherein the physiological parameter includes at least one of a blood glucose concentration, an insulin concentration, a body temperature, a distention of the stomach, a movement of the stomach, a stomach acid concentration, a gastric electrical activity, and a transabdominal impedance.

3. The method of claim 1, further comprising:
measuring a characteristic of the physiological parameter; and
controlling the degree of constriction of the gastric occluding device based on the measurement.

4. The method of claim 3, wherein the characteristic of the physiological parameter comprises at least one of a rate of change of the physiological parameter, an amplitude of the physiological parameter, a duration of the physiological parameter, an intensity of the physiological parameter and a concentration of the physiological parameter.

5. The method of claim 1, wherein controlling a degree of constriction of a gastric occluding device based on the monitored physiological parameter comprises:
comparing the monitored physiological parameter to criteria indicative of food intake; and
increasing the degree of constriction when the monitored physiological parameter satisfies the criteria indicative of food intake.

6. The method of claim 5, further comprising:
comparing the physiological parameter to criteria for reducing the degree of constriction; and
decreasing the degree of constriction when the monitored physiological parameter satisfies the criteria.

7. The method of claim 6, wherein comparing the physiological parameter to criteria for reducing the degree of constriction includes comparing the physiological parameter to a baseline value, and decreasing the degree of constriction when physiological parameter has returned to within a percentage of the baseline value.

8. The method of claim 6, wherein the gastric occluding device includes a gastric band.

9. A system comprising:
a sensor that monitors a physiological parameter that varies as a function of food intake;
a gastric occluding device positioned to restrict food intake by a patient; and
a control unit that controls a degree of constriction of the gastric occluding device based on the monitored physiological parameter.

10. The system of claim 9, wherein the gastric occluding device comprises an electromechanical gastric band.

11. The system of claim 10, wherein a diameter of the gastric band is adjusted via a micro motor that receives control signals from the control unit.

12. The system of claim 9, wherein the sensor comprises a chemical sensor.

13. The system of claim 10, wherein the chemical sensor senses at least one of blood glucose concentration, insulin concentration, and stomach acid concentration.

14. The system of claim 9, wherein the sensor comprises a mechanical sensor.

15. The system of claim 14, wherein the mechanical sensor senses at least one of motion of the stomach and distention of the stomach.

16. The system of claim 9, wherein the sensor comprises an electrical sensor.

17. The system of claim 16, wherein the electrical sensor senses at least one of gastric electrical activity and transabdominal impedance.

18. The system of claim 9, wherein the sensor comprises a temperature sensor.

19. The system of claim 9, wherein the control unit is implantable in the patient.

20. The system of claim 9, wherein the control unit is external to the patient, and wirelessly communicates with the sensor and the gastric occluding device.

21. The system of claim 9, wherein the control unit further measures a characteristic of the physiological parameter.

22. The system of claim 21, wherein the characteristic of the physiological parameter includes at least one of at least one of a rate of change of the physiological parameter, an amplitude of the physiological parameter, a duration of the physiological parameter, an intensity of the physiological parameter and a concentration of the physiological parameter.

23. The system of claim 9, wherein the control unit compares the monitored physiological parameter to criteria indicative of food intake to determine whether food has been ingested and increases the degree of constriction of the gastric occluding device when food has been ingested.

24. A system comprising:
a gastric occluding device positioned to restrict ingestion of food by a patient; and
a control unit that controls a degree of constriction of the gastric occluding device based on time.

25. The system of claim 24, wherein the control unit communicates a control signal causing an increase in a diameter of the gastric occluding device at preselected meal times.

26. The system of claim 25, wherein the control unit communicates a control signal to cause an increase in the diameter of the gastric occluding device for a predefined period of time.

27. A computer-readable medium comprising instructions that cause a processor to:
monitor a physiological parameter that varies as a function of food intake; and
control a degree of constriction of a gastric occluding device based on the monitored physiological parameter.

28. The computer-readable medium of claim 27, the instructions further causing the processor to monitor at least one of a blood glucose concentration, an insulin concentration, a body temperature, a distention of the stomach, a stomach acid concentration, a gastric electrical activity and a transabdominal impedance.
